(19) United States
(12) Patent Application Publication
(10) Pub. No.: US 2004/0131650 A1
(43) Pub. Date: Jul. 8, 2004

(54) Immunity adjuvant containing a complexed metal cation and vaccine containing same

(75) Inventors: Gerard Trouve, Castres (FR); Laurent Dupuis, Villejuif (FR)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(73) Assignee: SOCIETE D'EXPLOITATION DE PRODUITS POUR LES INDUSTRIES CHIMIQUES SEPPIC, PARIS CEDEX (FR)

(21) Appl. No.: 10/679,116

(22) Filed: Oct. 6, 2003

Related U.S. Application Data
(63) Continuation of application No. PCT/FR02/01057, filed on Mar. 27, 2002.

(30) Foreign Application Priority Data
Apr. 5, 2001 (FR) 01/04644

(51) Int. Cl. A61K 7/00

(52) U.S. Cl. 424/401

(57) ABSTRACT

The present invention relates to novel adjuvants for vaccine compositions and said compositions comprising at least one antigen, in particular an antigen of viral, bacterial or parasitic origin and at least one adjuvant. In particular, the subject of the invention is a composition comprising a fatty phase and a nonzero quantity of an organometallic gel comprising a complex of an anionic polymer, or a mixture of different anionic polymers, with a multivalent metal cation or a mixture of different multivalent metal cations.

This application is a continuation application of PCT International Application No. PCT/FR02/01057, filed Mar. 27, 2002, which claims benefit of priority from French Application No. 01/04644, filed Apr. 5, 2001. Both of these applications are incorporated herein by reference in their entirety.

### FIELD OF INVENTION

The present invention relates to novel adjuvants for vaccine compositions and said compositions comprising at least one antigen, in particular an antigen of viral, bacterial or parasitic origin and at least one adjuvant.

### BACKGROUND OF INVENTION

A great many substances are described as improving the immune response to an antigen.

There are water-insoluble inorganic salts among which aluminum hydroxide and calcium phosphate are the most 15 common and are the only ones authorized to date for human vaccination. They induce few reactions of intolerance at the site of vaccination but their efficacy is on the other hand poor and their effect of short duration.

There are also oils for injection which are used as adjuvants in veterinary vaccines. They are very effective but they sometimes induce local reactions. They are used as a mixture with the antigenic medium to form fluid emulsions for injection.

When these emulsions are of the oil-in-water (O/W) type, protection of the animal against the disease is provided rapidly, but only for a short duration, of the order of a few months.

When these emulsions are of the water-in-oil (W/O) type, the protection of the animal against the disease is only provided after a few weeks but it lasts for a long time, up to a year or more. It is thought that this long-term protection is due to the coating of the drops of antigenic medium with the oil.

There are also water-soluble salts of multivalent cations combined with an organic anion which have been described in French patents published under the numbers FR 2 733 151 and FR 2 754 715. These soluble salts are very well tolerated and provide rapid protection, but of a short duration, when they are used as sole adjuvant. When they are combined with oils in the form of (O/W) emulsions or microemulsions, they induce a prolonged protection for a period which is however less than that conferred by vaccines of the (W/O) type.

American patent published under the U.S. Pat. No. 3,925,544 and Belgian patent published under the U.S. Pat. Ser. No. 623 825 disclose vaccine compositions comprising, as adjuvant, from 1 to 5% weight/volume of sodium alginate and ions for insolubilizing the alginate, such as the calcium ion, the concentration of the sequestered ions for insolubilizing the alginate being less than the concentration necessary for forming a quantity of insoluble gel.

No vaccination means currently exists which makes it possible both to very rapidly provide protection against the disease and to maintain this protection for a long period. The applicant has therefore sought to solve this problem by developing an immunity adjuvant which does not have the abovementioned disadvantages.

### SUMMARY OF THE INVENTION

Accordingly, the subject of the invention is a composition comprising a fatty phase and a nonzero quantity of an organometallic gel comprising a complex of an anionic polymer, or a mixture of different anionic polymers, with a multivalent metal cation or a mixture of different multivalent metal cations.

In the composition which is the subject of the present invention, the organometallic gel can be a mixture of a volume Vc of a suspension or of a solution containing the multivalent cation salt or a mixture of multivalent cation salts with a volume Vp of a solution or of a suspension containing the anionic polymer or a mixture of anionic polymers in sufficient proportions to cause the gelling phenomenon leading to the organometallic gel, with, if necessary, stirring of the resulting mixture.

### DETAILED DESCRIPTION OF PREFERRED EMBODIMENTS

The fatty phase constituting the composition which is the subject of the present invention generally comprises oils of inorganic, plant or animal origin, alkyl esters of said oils, alkyl esters of fatty acids or alkyl ethers of fatty acids, esters of fatty acids and of polyols and ethers of fatty alcohols and of polyols.

As examples of oil of inorganic origin, there are the oils of petroleum origin, such as white mineral oils like MARCOL™ 52. As examples of oils of plant origin, there is peanut oil, olive oil, sesame oil, soybean oil, wheatgerm oil, grapeseed oil, sunflower oil, castor oil, linseed oil, soybean oil, corn oil, copra oil, palm oil, nut oil, hazelnut oil, rapeseed oil or alternatively squalane or squalene from olive. As examples of oils of animal origin, there is spermaceti oil, tallow oil, squalane or squalene extracted from fish livers.

As examples of alkyl esters of oils, there are methyl esters, ethyl esters, linear or branched propyl esters or linear or branched butyl esters of said oils.

As fatty acids which are appropriate for the preparation of the esters cited above, there are more particularly those containing from 12 to 22 carbon atoms, such as for example myristic acid, palmitic acid, oleic acid, ricinoleic acid or isostearic acid and advantageously a fatty acid which is liquid at 20° C.

As examples of fatty acid esters, there are the alkyl esters of fatty acids, such as ethyl oleate, methyl oleate, isopropyl myristate or octyl palmitate, esters of fatty acids and of polyols or ethers of fatty alcohols and of polyols, and more particularly fatty acid monoglycerides, fatty acid diglycerides, fatty acid triglycerides, esters of fatty acids with a polyglycerol or esters of fatty acids and of propylene glycol, esters of fatty acids with a hexol, such as for example sorbitol or mannitol, esters of fatty acids with a hexol anhydride, such as sorbitan or mannitan.

In the context of the present invention, the fatty phase may comprise only one of the compounds cited above or alternatively a mixture of several of the compounds cited above.

The composition which is the subject of the present invention generally comprises between about 5% and 70% by weight, and more particularly between 15% and 60% by weight of fatty phase.

Among the multivalent metal cations which can be complexed with the anionic polymer or the mixture of anionic polymers, there are mote particularly the divalent or trivalent metal cations and most particularly the divalent calcium, magnesium, manganese or zinc cations or alternatively the trivalent iron or aluminum cations.

In the suspension or the solution of cation salts, the organometallic gel contained in the composition which is the subject of the present invention, the concentration of metal cations [C], expressed in mol per liter of solution or suspension, is generally between about 10⁻³ mol per liter and 10 mol per liter, more particularly between 10⁻² mol per liter and 5 mol per liter and most particularly between 0.1 mol per liter and 1 mol per liter.

These cation salts are pharmaceutically acceptable. They are for example a hydroxide, a carbonate, a citrate, a gluconate, a glucoheptonate, a fructo-heptonate, a lactate, an acetate, a propionate, a salicylate, a chloride or a glycerophosphate.

As examples of salts used in the preparation of the organometallic gel of the composition which is the subject of the present invention, there is calcium hydroxide, magnesium carbonate, manganese carbonate, calcium gluconate, manganese gluconate, manganese glycerophosphate, zinc gluconate, calcium fructo-heptonate, aluminum salicylate or aluminum acetate.

According to a particular embodiment of the present invention, the multivalent cation salt used is manganese glycerophosphate or a mixture of manganese glycerophosphate and manganese gluconate.

Among the anionic polymers which can be complexed with the multivalent metal cations, there are more particularly the sulfated polymers, dextran, carrageenans, catboxylic polymers, polyacrylates, pectins, alginates, natural gums, xanthan gum or guar gum.

According to a particular embodiment of the present invention, the anionic polymer used is a sodium alginate.

In the suspension or solution of anionic polymers of the organometallic gel contained in the composition which is the subject of the present invention, the concentration of anionic polymers [P], expressed as a percentage by weight of the solution or of the suspension, is generally between about 0.1% and 10% by weight, more particularly between 0.5% and 5% by weight and most particularly between 1% and 5% by weight.

The proportions of suspension or solution of cation salt and of solution or suspension of anionic polymer for preparing the mixture leading to the production of the organometallic gel are chosen such that the [P]/[C] ratio is between 0.01 and 100, more particularly between 0.1 and 50 and most particularly between 1 and about 10.

The solvents of said suspensions or solutions used to prepare the organometallic gel are generally polar solvents and preferably miscible with each other. They are preferably water or a pharmaceutically acceptable aqueous-alcoholic mixture.

According to a particular aspect of the present invention, the organometallic gel can be a mixture of an aqueous suspension or solution containing the multivalent cation salt or a mixture of multivalent cation salts with an aqueous solution or suspension containing the anionic polymer or the mixture of anionic polymers, with, if necessary, stirring of the resulting mixture.

According to another particular aspect of the present invention, the organometallic gel can be a mixture of an aqueous suspension or solution containing a multivalent cation salt with an aqueous solution or suspension containing an anionic polymer, with, if necessary, stirring of the resulting mixture.

The composition as defined above is preferably in the form of an emulsion and in particular in the form of an emulsion whose continuous phase is the fatty phase and the dispersed phase the multivalent metal cation-anionic polymer gelled complex.

The composition as defined above may also comprise one or more pharmaceutically acceptable surfactants.

Among the surfactants used in the composition which is the subject of the present invention, there are nonionic surfactants, for example esters of polyglycerols, esters of sugars such as esters of sorbitan, mannitan or sucrose, esters of ethoxylated sugars, alkoxylated fatty alcohols, ethoxylated fatty acids, monoglycerides and diglycerides modified by reaction with acetic acid or lactic acid; ethoxylated monoglycerides, diglycerides or triglycerides, ethers of sugars, such as glucose ethers, xylose ethers and lactitol ethers.

The surfactants used are more particularly chosen such that the hydrophilic-lipophilic balance (HLB) of the mixture of surfactants is between 4 and 12, and preferably between 5 and 8.

The composition as defined above generally comprises between about 0.5% and 10% by weight and preferably between 1% and 5% by weight of surfactants.

The subject of the invention is also a method for preparing the emulsion as defined above, comprising the following steps:

(a) preparing an aqueous suspension or solution containing at least one insoluble multivalent cation salt, at least one water-soluble anionic polymer and optionally at least one hydrophilic surfactant;

(b) emulsifying the suspension prepared in step a), with an oily phase optionally containing a lipophilic surfactant;

(c) if necessary, solubilizing the insoluble multi-valent cation salt by modifying the pH of the emulsion;

(d) optionally adding an excess of multivalent cation; and

(e) neutralizing the final emulsion obtained.

Step (a) of the method generally consists in mixing a volume Vc of a suspension or solution of cation salt with a volume Vp of a solution or suspension of anionic polymer, in a Vc/Vp volume ratio generally of between 1/100 and 1/1, preferably between 1/50 and 1/10, either by pouring the suspension or solution of cation salt into the solution or suspension of anionic polymer with, if necessary, stirring of the resulting mixture, or by pouring the suspension or solution of anionic polymer into the solution or suspension of cation salt with, if necessary, stirring of the resulting mixture.

There are preferably used in step (a) one or more salts comprising calcium hydroxide, magnesium carbonate, manganese carbonate, calcium gluconate, manganese gluconate, manganese glycerophosphate, zinc gluconate, calcium fructoheptonate, aluminum salicylate or aluminum acetate.

According to a particular variant of the method as defined above, the emulsion obtained in step (e) is dissolved in a solvent of the fatty phase in order to obtain a suspension of organometallic gel and the resulting suspension is subjected to centrifugation in order to isolate said gel. This variant is used to prepare a composition with a low oil content.

According to another aspect of the present invention, its subject is the use of the composition as defined above as adjuvant phase of a vaccine composition.

The subject of the invention is also a method for preparing a vaccine comprising the addition, as immunity adjuvant, of an effective quantity of the composition as defined above.

The composition as defined above may be used in combination with conventional oily adjuvants known to persons skilled in the art.

When the vaccine prepared is of the W/O emulsion type, the composition which is the subject of the present invention is mixed with the antigenic phase and then the whole is emulsified.

According to a final aspect of the present invention, its subject is a composition comprising at least one antigen or at least one generator in vivo of a compound comprising an amino acid sequence and a nonzero quantity of a composition as defined above.

The expression antigen or at least one generator in vivo of a compound comprising an amino acid sequence denotes either killed microorganisms such as viruses, bacteria or parasites, or purified fractions of these microorganisms, or live microorganisms whose pathogenicity has been attenuated. By way of examples of viruses which can constitute an antigen according to the present invention, there are the rabies virus, the herpesviruses, such as the Aujeszky disease virus, orthomixoviruses such as Influenzae, picornaviruses such as the foot-and-mouth disease virus or retro-viruses such as HIVs. By way of microorganism of the bacterial type which can constitute an antigen according to the present invention, there may be mentioned E. coli, and those of the genera Pasteurella, Staphylococcus and Furonculosis, Vibriosis, Streptococcus. By way of examples of parasites, there are those of the genera Trypanosoma, Plasmodium and Leishmania. There may also be mentioned recombinant viruses, in particular nonenveloped viruses such as adenoviruses, vaccinia virus, canarypox virus, herpes-viruses or baculoviruses. It is also understood to mean a live nonenveloped recombinant viral vector whose genome contains, preferably inserted into a part not essential for the replication of the corresponding enveloped virus, a sequence encoding an antigenic subunit inducing an antibody synthesis and/or a protective effect against the abovementioned enveloped virus or pathogenic mcroorganism; these antigenic subunits may be for example a protein, a glycoprotein, a peptide or a peptide fraction and/or a fraction which protects against an infection by a live microorganism such as an enveloped virus, a bacterium or a parasite. The exogenous gene inserted into the microorganism may be for example derived from an Aujeszky virus or HIV.

There may be mentioned in particular a recombinant plasmid comprising a nucleotide sequence into which is inserted an exogenous nucleotide sequence obtained from a pathogenic microorganism or virus. The latter nucleotide sequence is intended to allow the expression of a compound comprising an amino acid sequence, this compound itself being intended to trigger an immune reaction in a host organism.

The expression generator “in vivo” of a compound comprising an amino acid sequence denotes a whole biological product capable of expressing said compound in the host organism into which said generator in vivo is introduced. The compound comprising the amino acid sequence may be a protein, a peptide or a glycoprotein. These generators in vivo are generally obtained by methods derived from genetic engineering. More particularly, they may consist of live microorganisms, generally a virus, playing the role of a recombinant vector, into which is inserted a nucleotide sequence, in particular an exogenous gene. These compounds are known as such and are used in particular as recombinant subunit vaccine. In this regard, reference may be made to the article by M. ELOIT et al., Journal of Virology (1990) 71, 2925-2431 and to international patent applications published under the numbers WO-A-91/00107 and WO-A-94/16681. The generators in vivo according to the invention may also consist of a recombinant plasmid comprising an exogenous nucleotide sequence capable of expressing, in a host organism, a compound comprising an amino acid sequence. Such recombinant plasmids and their mode of administration into a host organism were described in 1990 by LIN et al., Circulation 82: 2217, 2221; COX et al., J. of VIROL, September 1993, 67, 9, 5664-5667 and in international application published under the number WO 95/25542. According to the nature of the nucleotide sequence contained in the generator in vivo, the compound comprising the amino acid sequence which is expressed in the host organism may:

(i) be an antigen, and allow the initiation of an immune reaction,

(ii) have a curative action on a disease, essentially a disease of a functional order, which is triggered in the host organism. In this case, the generator in vivo allows a treatment of the host, of the gene therapy type.

By way of example, such a curative action may consist of a synthesis by the generator in vivo of cytokines, such as interleukins, in particular interleukin 2. These allow the initiation or the enhancement of an immune reaction aimed at the selective elimination of cancer cells.

A composition according to the invention comprises a concentration of antigen which depends on the nature of this antigen and on the nature of the treated subject. It is however particularly remarkable that an adjuvant according to the invention makes it possible to substantially reduce the usual dose of antigen required. The appropriate concentration of antigen may be determined conventionally by persons skilled in the art. Generally, this dose is of the order of 0.1 μg/cm³ to 1 g/cm³, more generally between 1 μg/cm³ and 100 mg/cm³. The concentration of said generator in vivo in the composition according to the invention depends, here again, in particular on the nature of said generator and the host to which it is administered. This concentration can be easily determined by persons skilled in the art on the basis of a routine experiment. As a guide, it is however possible to specify that when the generator in vivo is a recombinant microorganism, its concentration in the composition according to the invention may be between 10² and 10¹⁵ microorganisms/cm³, preferably between 10⁵ and 10¹² microorganisms/cm³. When the generator m vivo is a recombinant plasmid, its concentration in the composition according to the invention may be between 0.01 g/dm³ and 100 g/dm³. The vaccine as defined above is prepared by mixing the adjuvant phase and the antigenic phase, by optionally adding water or a pharmaceutically acceptable diluent medium. The following examples illustrate the invention without however limiting it.

### EXAMPLES

### Example 1

A solution containing 1% of sodium alginate of high viscosity and a high content of guluronic acid (SATIALGINE™ SG800) is prepared.

A 500 millimolar aqueous suspension of an insoluble salt of a water-insoluble salt, calcium hydroxide, is prepared. 1 ml of the suspension and 20 g of the sodium alginate solution are mixed. The mixture obtained is dispersed by means of a quick stirrer in 100 g of a white mineral oil (MARCOL™ 52) containing 1% by weight of a lipophilic surfactant, sorbitan monooleate or MONTANE™ 80, having an HLB number equal to about 4.3.

An emulsion is obtained which is acidified with a few drops of concentrated acetic acid. This emulsion has a continuous oil phase; its dispersed phase comprises a stable gelled complex of calcium alginate.

This calcium alginate emulsion constitutes an immunity adjuvant, which may be emulsified with an antigenic medium to form a stable, W/O type vaccine with improved efficacy. This novel immunity adjuvant may be optionally mixed with another oily adjuvant such as those of the family of MONTANIDE™ ISA adjuvants marketed by the company Seppic before manufacture of the final vaccine.

### Example 2

A solution containing 3.5% of sodium alginate of low viscosity and a high content of guluronic acid (SATIALGINE™ S80) is prepared.

A 500 millimolar aqueous suspension of an insoluble salt, manganese carbonate, is prepared.

1 ml of the suspension and 20 g of the sodium alginate solution are mixed. The mixture obtained is dispersed in 100 g of MARCOL™ 52 containing 2% by weight of MONTANE™ 80, by means of a fast stirrer revolving at 3,000 revolutions/min for 3 minutes.

An emulsion is obtained which is acidified with a few drops of concentrated acetic acid in order to solubilize the manganese carbonate.

An imunity adjuvant is thus obtained which is an emulsion, contains a continuous oil phase and whose dispersed phase consists of a stable gelled complex of manganese alginate.

### Example 3

A solution containing 3.5% of sodium alginate of low viscosity and a high content of guluronic acid (SATIALGINE™ S80) is prepared.

A 500 millimolar suspension of a sparingly soluble salt, manganese glycerophosphate, is prepared.

1 ml of the suspension, 20 ml of the alginate solution and 1.05 g (5%) of a hydrophilic surfactant, of polyethoxylated sorbitan oleate (EO value=80), MONTANOX™ 80 having an HLB number equal to 15 are mixed. The mixture obtained is dispersed in 100 g of MARCOL™ 52, containing 5% by weight of MONTANE™ 80, by means of a fast stirrer revolving at 3 000 revolutions/min for 3 minutes. The HLB number for the surfactant system used (MONTANOX™ 80+MONTANE™ 80) is 6. An emulsion is obtained which is acidified with a few drops of concentrated acetic acid in order to solubilize the manganese glycerophosphate and to form the complex of manganese alginate which is then neutralized to a pH equal to 5.5 with sodium hydroxide. The adjuvant thus obtained is an emulsion whose continuous phase is the oily phase and whose dispersed phase comprises a stable gelled complex of manganese alginate.

The efficacy of this adjuvant is evaluated in female mice of the OF1 strain weighing 20 grams, into which there are injected subcutaneously 100 μl of vaccines containing ovalbumin grade V (OVA), as antigen (all the preparations were adjusted so that the dose of antigen administered per animal is constant and equal to 1 μg per injection). The vaccination scheme comprises a booster 28 days after the first injection.

A first group of mice receives an OVA dose alone without adjuvant (control 1).

A second group of mice receives a vaccine (A) of the W/O type (preparation A), comprising a portion of standard oily adjuvant (MONTANIDE198 ISA 564, marketed by the company SEPPIC) and of a portion of OVA in saline (composition according to the state of the art).

A third group of mice receives a preparation (B) comprising three portions of vaccine (A) for 1 portion of adjuvant containing a complex of manganese alginate prepared as described above (composition according to the invention).

A fourth group of mice receives a preparation (C) comprising a portion of vaccine (A) for a portion of adjuvant containing a complex of manganese alginate prepared as described above (composition according to the invention).

The levels of IgG1 and IgG2 antibodies are measured at D=28 days, just before the booster at D=56 days and at D=90 days. The results are presented in the following table.

TABLE 1

| | IgG1 | | | IgG2a | | |
|---|---|---|---|---|---|---|
| Vaccine | D28 | D56 | D90 | D28 | D56 | D90 |
| Control 1 | 100 | 1,000 | 100 | 100 | 1,000 | 100 |
| Preparation (A) | 2,400 | 32,000 | 64,000 | 100 | 1,000 | 2,400 |
| Preparation (B) | 12,800 | 12,800 | 96,000 | 1,600 | 8,000 | 12,800 |
| Preparation (C) | 64,000 | 64,000 | 96,000 | 2,400 | 16,000 | 24,000 |

The results show that the addition of the complex of manganese alginate markedly increases the efficacy of the standard W/O vaccine (A) in the short term (28 days) both in the humoral response (IgG1) , and in the cellular response (IgG2a). A similar effect is observed after the booster, at 56 days and at 90 days.

### Example 4

An immunity adjuvant comprising a complex of manganese alginate emulsified in mineral oil is prepared as in Example 3.

A portion of this adjuvant is mixed with a portion of ovalbumin solution in saline in order to obtain an intermediate preparation (I).

A placebo emulsion (P) comprising a portion of standard adjuvant MONTANIDE™ ISA 564 and a portion of saline is prepared.

The efficacy of this adjuvant is evaluated in female mice of the OF1 strain weighing 20 grams, into which are injected subcutaneously 100 μl of vaccines containing ovalbumin grade V (OVA) as antigen (all the preparations were adjusted so that the antigen dose administered per animal is constant and equal to 1 μg per injection). The vaccination scheme comprises a booster 28 days after the first injection.

A first group of mice receives a dose of OVA alone without adjuvant (control 1).

A second group of mice receives a vaccine (A) of the W/O type (preparation A) comprising a portion of MONTANIDE™ ISA 564 and a portion of OVA in saline (composition according to the state of the art).

A third group of mice receives a preparation (D) comprising three portions of placebo (P) for a portion of preparation (I) (composition according to the invention).

A fourth group of mice receives a preparation (E) comprising a portion of placebo (P) for a portion of preparation (I) (composition according to the invention).

The levels of IgG1 and IgG2 antibodies are measured at D=28 days, just before the booster at D=56 days and at D=90 days. The results are presented in the following table.

TABLE 2

| | IgG1 | | | IgG2a | | |
|---|---|---|---|---|---|---|
| Vaccine | D28 | D56 | D90 | D28 | D56 | D90 |
| Control 1 | 100 | 1,000 | 100 | 100 | 1,000 | 100 |
| Preparation (A) | 2,400 | 32,000 | 64,000 | 100 | 1,000 | 1,000 |
| Preparation (D) | 12,800 | 128,000 | 128,000 | 200 | 6,000 | 12,800 |
| Preparation (E) | 12,800 | 128,000 | 128,000 | 200 | 9,600 | 12,800 |

The results presented in Table 2 show a marked improvement in the efficacy of the vaccine containing a complex of manganese alginate in the short term ( D=28 days) and after the booster. The levels of IgG1 antibody are not significantly different from those obtained by the method of preparing the vaccine of Example 3. This demonstrates that the adjuvants according to the invention are effective, regardless of their method of use, (addition to a standard oily vaccine as in Example 3 or alternatively addition to an antigenic medium followed by addition to a standard oily adjuvant as in Example 4).

### Example 5

The complex of emulsified manganese alginate obtained in Example 3 is diluted by half an organic solvent (ether or isopropyl alcohol). A portion of the mineral oil of the emulsion is dissolved and the beads of alginate complex are isolated by centrifugation. The solvent residue is evaporated and an immunity adjuvant enriched with complex containing only about 5% of residual mineral oil is obtained.

The efficacy of this adjuvant is evaluated in female mice of the OF1 strain weighing 20 grams, into which are injected subcutaneously 100 μl of vaccines containing ovalbumin grade V (OVA) as antigen (all the preparations were adjusted so that the antigen dose administered per animal is constant and equal to 1 μg per injection). The vaccination scheme comprises a booster 28 days after the first injection.

A first group of mice receives a dose of OVA alone without adjuvant (control 1).

A second group of mice receives a vaccine containing, as adjuvant, manganese glycerophosphate such that the concentration of Mn⁺⁺ cation is the same as that of the preparation F and containing the same quantity of OVA as the preparation F (control 2) (composition according to the state of the art).

A third group of mice receives a preparation (F) comprising the mixture of adjuvant, enriched with a complex of manganese alginate with an antigenic solution of OVA in order to form a vaccine preparation (F) containing 10 pg/ml of albumin.

The levels of IgG1 and IgG2 antibodies are measured at D=28 days, just before the booster at D=56 days and at D=90 days. The results are presented in the following table.

TABLE 3

| | IgG1 | | | IgG2a | | |
|---|---|---|---|---|---|---|
| Vaccine | D28 | D56 | D90 | D28 | D56 | D90 |
| Control 1 | 100 | 1,000 | 100 | 100 | 1,000 | 100 |
| Control 2 | 12,800 | 38,000 | 10,000 | 100 | 1,000 | 1,000 |
| Preparation (F) | 2,000 | 12,000 | 20,000 | 100 | 1,000 | 2,000 |

The delay effect of the adjuvant, “complex of manganese alginate”, relative to the control 2, noncomplexed cation, is clearly demonstrated by the assays of antibodies.

### Example 6

100 g of a solution containing 3.5 g of sodium alginate, 1.13 g of manganese glycerophosphate and 5.7 mg of OVA ate prepared. 60 g of the mixture obtained are dispersed, by means of a fast stirrer revolving at 3 000 revolutions/min, in 100 g of MARCOL™ 52 containing 5% by weight of a mixture of mannitan monooleate and polyethoxylated oleic acid in a proportion such that the HLB number of the mixture is equal to 6.

An emulsion is obtained which is acidified with a few drops of concentrated acetic acid in order to solubilize the manganese glycerophosphate and to form the complex of manganese alginate.

The emulsion is then brought to pH=5.5 by adding triethanolamine. A vaccine (G) is obtained which consists of the OVA antigen and an oily adjuvant composed of an oil and a complex of manganese alginate.

The efficacy of this adjuvant is evaluated in female mice of the OF1 strain weighing 20 grams, into which are injected subcutaneously 100 μl of vaccines containing ovalbumin grade V (OVA) as antigen (all the preparations were adjusted so that the antigen dose administered per animal is constant and equal to 1 μg per injection). The vaccination scheme comprises a booster 28 days after the first injection.

A first group of mice receives a dose of OVA alone without adjuvant (control 1).

A second group of mice receives a vaccine (A) of the W/O type (preparation A) comprising a portion of MONTANIDE™ ISA 564 and a portion of OVA in saline (composition according to the state of the art).

A third group of mice receives the vaccine (G) (composition according to the invention).

A fourth group of mice receives a preparation (H) comprising a portion of the placebo (P) prepared in Example 4 and a portion of vaccine (G) (composition according to the invention).

The levels of IgG1 and IgG2 antibodies are measured at D=28 days immediately before the booster at D=56 days and at D=180 days. The results are presented in the following table.

TABLE 4

| | IgG1 | | | IgG2a | | |
|---|---|---|---|---|---|---|
| Vaccine | D28 | D56 | D90 | D28 | D56 | D90 |
| Control 1 | 100 | 1,000 | 100 | 100 | 1,000 | 100 |
| Preparation (A) | 2,400 | 32,000 | 8,000 | 100 | 1,000 | 1,000 |
| Preparation (G) | 4,800 | 32,000 | 4,800 | 100 | 2,000 | 2,000 |
| Preparation (H) | 12,800 | 128,000 | 12,800 | 3,200 | 8,000 | 4,800 |

The vaccine G containing the emulsified complex as adjuvant is more effective than the standard vaccine A in the short tern and has a similar efficacy in the long term.

The vaccine H containing a mixture of two adjuvants is markedly more effective than the two vaccines with a single adjuvant both in the short term and after 56 days. A synergy is therefore observed.

### Example 7

The emulsified complex of manganese alginate obtained in Example 3 is used and it is diluted by half in an organic solvent (ether or isopropyl alcohol). A portion of the mineral oil of the emulsion is dissolved and the beads of alginate complex may be isolated by centrifugation.

The solvent residue is evaporated and an immunity adjuvant enriched with complex containing only about 5% of residual mineral oil is obtained.

The efficacy of this adjuvant is evaluated in female mice of the OF1 strain weighing 20 grams into which are subcutaneously injected 100 μl of vaccines containing a parasitic antigen of Trichinella spiralis larvae (all the preparations were adjusted so that the antigen dose administered per animal is constant and equal to 5 μg per injection). The vaccination scheme comprises a booster 28 days after the first injection.

A first group of mice receives a vaccine of the W/O type comprising a portion of MONTANIDE™ ISA 763 and a portion of parasitic antigen of Trichinella spiralis larvae in saline (control 4).

A second group of mice receives a vaccine containing, as adjuvant, manganese glycerophosphate such that the concentration of Mn⁺⁺ cation is the same as that of the preparation J and containing the same quantity of parasitic antigen of Trichinella spiralis larvae as the preparation J (control 5).

A third group of mice receives a preparation (J) containing 50 μg/ml of antigen comprising the mixture of adjuvant enriched with a complex of manganese alginate with the antigenic solution of parasitic antigen of Trichinella spiralis larvae (composition according to the invention).

The levels of IgG1 and IgG2 antibodies are measured at D=14 days, D=42 days and D=90 days. The results are presented in the following table.

TABLE 5

| | IgG1 | | | IgG2a | | |
|---|---|---|---|---|---|---|
| Vaccine | D14 | D42 | D90 | D14 | D42 | D90 |
| Control 4 | 1,000 | 100,000 | 64,000 | 30 | 10,000 | 5,000 |
| Control 5 | 1,000 | 64,000 | 10,000 | 50 | 1,000 | 1,000 |
| Preparation (J) | 1,000 | 100,000 | 64,000 | 200 | 20,000 | 2,000 |

The results show that the vaccine according to the invention is as effective in the short term as the vaccine containing the soluble salt and that it is more effective than the vaccine of the W/O type. In the long term, it is almost as effective as the oily W/O vaccine and more effective than the vaccine with a soluble salt.

### Example 8

The vaccines containing the different adjuvants described in Examples 1 to 6 are subcutaneously injected into mice of the OF1 strain (volume injected: 100 μl). The intensity of the local reactions at the site of injection is noted after seven days, on a numerical scale ranging from 0 (no reaction) to 5 (very strong reaction with necrosis of the tissue), after 7 days. The results presented in the following table show that the vaccines containing the adjuvants according to the invention are well tolerated, the local reactions not exceeding that for the control A.

| Adjuvant | A | B | C | D | E | F | G | H |
|---|---|---|---|---|---|---|---|---|
| Score | 1.6 | 1.3 | 1.6 | 1.0 | 1.6 | 1.5 | 0.8 | 1.5 |

1. A composition comprising:
(i) a fatty phase; and
(ii) a nonzero quantity of an organometallic gel, wherein the organometallic gel comprises a complex of an anionic polymer, or a mixture of different anionic polymers, with a multivalent metal cation or a mixture of different multivalent metal cations.

2. The composition of claim 1, wherein the organometallic gel comprises a mixture of a volume Vc of a suspension, or solution, comprising multivalent cation salt or a mixture of multivalent cation salts, with a volume Vp of suspension, or solution, comprising anionic polymer or a mixture of anionic polymers.

3. The composition of claim 1, in which the fatty phase comprises oils of inorganic, plant or animal origin; alkyl esters of said oils; alkyl esters of fatty acids; alkyl ethers of fatty acids; esters of fatty acids and of polyols; ethers of fatty alcohols and of polyols; or a combination thereof.

4. The composition of claim 3, wherein the alkyl esters of oils comprise methyl esters, ethyl esters, linear or branched propyl esters, linear or branched butyl esters of said oils, or a combination thereof.

5. The composition of claim 3, wherein the oils comprise white mineral oils, peanut oil, olive oil, sesame oil, soybean oil, wheatgerm oil, grapeseed oil, sunflower oil, castor oil, linseed oil, soybean oil, corn oil, copra oil, palm oil, nut oil, hazelnut oil, rapeseed oil, squalane or squalene from olive or extracted from fish livers, or a combination thereof.

6. The composition of claim 3, wherein the alkyl esters of fatty acids comprise fatty acid esters comprising 12 to 22 carbon atoms.

7. The composition of claim 3, wherein the alkyl esters of fatty acids comprise ethyl oleate, methyl oleate, isopropyl myristate, octyl palmitate, or a combination thereof.

8. The composition of claim 3, wherein the esters of fatty acids and of polyols comprise fatty acid monoglycerides, fatty acid diglycerides, fatty acid triglycerides, esters of fatty acids with a polyglycerol, esters of fatty acids and of propylene glycol, esters of fatty acids with a hexol, or a combination thereof.

9. The composition of claim 1, wherein the composition comprises between about 5% and 70% by weight of fatty phase.

10. The composition of claim 1, wherein the multivalent metal cation, or mixture of multivalent metal cations, comprises divalent, trivalent metal cations, or a combination thereof.

11. The composition of claim 2, wherein the suspension, or solution, of multivalent cation salt or mixture of multivalent cation salts, comprises a concentration of metal cations [C], expressed in mol/liter of solution or suspension, between about 10⁻³ and 10 mol/liter.

12. The composition of claim 2, wherein the multivalent cation salt, or mixture of multivalent cation salts, comprises hydroxides, carbonates, citrates, gluconates, glucoheptonates, fructoheptonates, lactates, acetates, propionates, salicylates, chlorides, or glycerophosphates, of said cations, or a combination thereof.

13. The composition of claim 12, wherein the multivalent cation salt, or mixture of multivalent cation salts, comprises calcium hydroxide, magnesium carbonate, manganese carbonate, calcium gluconate, manganese gluconate, manganese glycerophosphate, zinc gluconate, calcium fructoheptonate, aluminum salicylate, aluminum acetate, or a combination thereof.

14. The composition of claim 13, wherein the multivalent cation salt, or mixture of multivalent cation salts, comprises manganese glycerophosphate or a mixture of manganese glycerophospate and manganese gluconate.

15. The composition of claim 2, wherein the anionic polymer, or mixture of anionic polymers, comprises sulfated polymers, dextran, carrageenans, carboxylic polymers, polyacrylates, pectins, alginates or natural gums, xanthan gum, guar gum, or a combination thereof.

16. The composition of claim 2, wherein the anionic polymer, or mixture of anionic polymers, comprises sodium alginate.

17. The composition of claim 2, wherein the suspension, or solution, of anionic polymer, or mixture of anionic polymers, comprises a concentration of anionic polymers [P], expressed as a percentage by weight of the suspension or solution, between about 0.1% and 10% by weight.

18. The composition of claim 2, wherein the [P]/[C] ratio is between about 0.01 and 100.

19. The composition of claim 2, further comprising at least one solvent of said suspensions, or solutions, said solvent comprising water.

20. The composition of claim 19, wherein the organometallic gel comprises a mixture of an aqueous suspension, or solution, comprising a multivalent cation salt with an aqueous solution, or suspension, comprising an anionic polymer.

21. The composition of claim 1, wherein said composition is in the form of an emulsion with a continuous phase comprising the fatty phase and a dispersed phase comprising the multivalent metal cation-anionic polymer gelled complex.

22. The composition of claim 1, wherein said composition further comprises one or more pharmaceutically acceptable surfactants.

23. The composition of claim 22, wherein the one or more surfactants comprise nonionic surfactants.

24. The composition of claim 22, wherein the one or more surfactants comprise a hydrophilic-lipophilic balance (HLB) between about 4 and 12.

25. The composition of claim 22, wherein the composition comprises between about 0.5% and 10% by weight of the one or more surfactants.

26. A method for preparing an emulsion, comprising:
(a) preparing an aqueous suspension, or solution, containing at least one water-soluble anionic polymer, at least one hydrophilic surfactant, or combination thereof;
(b) emulsifying the suspension prepared in step (a) with an oily phase; and
(c) neutralizing the final emulsion obtained.

27. The method of claim 26, wherein step (a) further comprises mixing a volume Vc of a suspension, or solution, of cation salt with a volume Vp of a solution, or suspension, of anionic polymer, in a Vc/Vp volume ratio of between 1/100 and 1/1, either by pouring the suspension, or solution, of cation salt into the solution, or suspension, of anionic polymer, or by pouring the solution, or suspension, of anionic polymer into the solution, or suspension, of cation salt.

28. The method of claim 26, wherein said method further comprises dissolving the neutralized final emulsion of step (c) in a solvent of a fatty phase to obtain a suspension of organometallic gel and centrifuging the suspension of organometallic gel to isolate the organometallic gel.

29. A method for preparing a vaccine, comprising adding, as immunity adjuvant, an effective quantity of the composition of claim 1.

30. A composition comprising at least one antigen or at least one generator in vivo of a compound comprising an amino acid sequence and a nonzero quantity of the composition of claim 1.

31. The composition of claim 2, wherein the volume Vc and the volume Vp are in sufficient proportions to cause the gelling phenomenon of the organometallic gel.

32. The composition of claim 6, wherein the fatty acid esters comprise esters of myristic, palmitic, oleic, ricinoleic and isostearic acids.

33. The composition of claim 8, wherein the esters of fatty acids and polyols comprise esters of fatty acids with sorbitol or mannitol, and esters of fatty acids with hexol anhydride.

34. The composition of claim 8, wherein the esters of fatty acids and polyols comprise esters of fatty acids with sorbitan or mannitan.

35. The composition of claim 9, wherein the composition comprises between about 15% and 60% by weight of fatty phase.

36. The composition of claim 10, wherein the divalent metal cations comprise divalent cations of calcium, magnesium, manganese, zinc, or a combination thereof.

37. The composition of claim 10, wherein the trivalent metal cations comprise trivalent cations of iron, aluminum, or a combination thereof.

38. The composition of claim 11, wherein [C] is between about 10⁻² and 5 mol/liter.

39. The composition of claim 38, wherein [C] is between about 0.1 and 1 mol/liter.

40. The composition of claim 17, wherein [P] is between about 0.5% and 5% by weight.

41. The composition of claim 40, wherein [P] is between about 1% and 5% by weight.

42. The composition of claim 18, wherein the [P]/[C] ratio is between about 0.1 and 50.

43. The composition of claim 42, wherein the [P]/[C] ratio is between about 1 and 10.

44. The composition of claim 23, wherein the surfactants comprise nonionic surfactants from esters of polyglycerols, esters of sugars, esters of ethoxylated sugars, alkoxylated fatty alcohols, ethoxylated fatty acids, monoglycerides and diglycerides modified by reaction with acetic acid or lactic acid, ethoxylated monoglycerides, diglycerides or triglycerides, ethers of sugars, or a combination thereof.

45. The composition of claim 45, wherein the esters of sugars comprise esters of sorbitan, mannitan, sucrose, or a combination thereof.

46. The composition of claim 45, wherein the ethers of sugars comprise glucose ethers, xylose ethers, lactitol ethers, or a combination thereof.

47. The composition of claim 24, wherein the one or more surfactants comprise an HLB between about 5 and 8.

48. The composition of claim 25, wherein the composition comprises between about 1% and 5% by weight of the one or more surfactants.

49. The method of claim 26, wherein the oily phase comprises a lipophilic surfactant.

50. The method of claim 26, further comprising, following step (b), solubilizing an insoluble multivalent cation salt by modifying the pH of the emulsion.

51. The method of claim 26, further comprising, following step (b), adding an excess of multivalent cation.

52. The method of claim 27, wherein the Vc/Vp volume ratio is between about 1/50 and 1/10.
